(19) United States
(12) Patent Application Publication
(10) Pub. No.: US 2020/0345656 A1
(43) Pub. Date: Nov. 5, 2020

(54) CANNABINOIDS COMPOSITIONS AND METHODS

(71) Applicant: YISSUM RESEARCH DEVELOPMENT COMPANY OF THE HEBREW UNIVERSITY OF JERUSALEM LTD., Jerusalem (IL)

(72) Inventors: Elka TOUITOU, Hod Hasharon (IL); Hiba NATSHEH, Jerusalem (IL)

(21) Appl. No.: 16/767,799

(22) PCT Filed: Nov. 4, 2018
(86) PCT No.: PCT/IL2018/051175

§ 371 Date: May 28, 2020

Related U.S. Application Data
(60) Provisional application No. 62/591,782, filed on Nov. 29, 2017.

(51) Int. Cl. A61K 31/05; A61K 47/24; A61K 47/10; A61K 47/44; A61K 9/00; A61P 23/00

(52) U.S. Cl. CPC A61K 31/05; A61K 47/24; A61K 47/10; A61P 23/00; A61K 9/0043; A61K 9/0031; A61K 9/0034; A61K 47/44

(57) ABSTRACT

A composition for administration into the nasal cavity, the rectal cavity and the vaginal cavity, wherein the composition is an essentially non-aqueous composition comprising: not less than 25% by weight of one or more cannabinoid(s); from 25% to 55% by weight one or more phospholipid(s); and optionally one or more antioxidant(s).

It has long been recognized that the active ingredients of cannabis are able to provide relief for a variety of symptoms and conditions, for example, to reduce pain. The major components include cannabidiol (CBD), tetrahydrocannabinol (THC) and cannabinol (CBN). The term “cannabinoid”, as used herein, is meant to include compounds interacting with cannabinoid receptors, either naturally occurring or synthetic compounds, e.g., each of the aforementioned components, derivatives and analogues thereof, as described further below.

Cannabinoids are generally difficult to formulate into pharmaceutical dosage dorms, largely due to their strong lipophilic character, indicated by their high log P values (octanol/water partition). In particular, there exists a need in intranasal cannabinoid liquid (spray or drops) or gel-like intranasal cannabinoid formulation with high cannabinoids content. Additionally, cannabinoids-rich, rectally or vaginally administrable compositions, are also sought to offer various benefits, as shown below.

Cannabinoids-containing nasally administrable compositions were demonstrated in U.S. Pat. No. 6,383,513 (for example, THC was dissolved in sesame oil, and the oily phase was dispersed in water in the presence of phospholipid as an emulsifier); U.S. Pat. No. 8,911,751 (in the name of the present inventor; the drug was delivered in a vesicular carrier consisting of a suitably proportioned mixture of water, ethanol, propylene glycol and phospholipids); WO 2016/144376 (reporting the incorporation of 20% by weight THC+CBD mixture into a carrier comprising phospholipids, oil and water); and US 2018/0042845 (again based on emulsion formulation; it is reported that the amount of cannabinoids loaded in the oily fraction is up to 50).

A different approach towards cannabinoids formulations was recently presented in WO 2017/098502, where it was shown that mixtures consisting of cannabinoids and preferably not less 60% by weight phospholipids create compact masses that can be easily processed and shaped into dosage forms suitable for oral delivery. In the formulations tested in WO 2017/098502, phospholipids generally constitute the major component; for instance, in Example 6 of WO 2017/098502, it is reported that solid compositions consisting of cannabinoids and phospholipids at weight ratios of 1:9, 3:7 and 4:6 were subjected to disintegration tests.

We have now found that suitably proportioned mixtures of cannabinoids and phospholipids can be used to create non-aqueous, non-solid pharmaceutical preparations with high cannabinoids concentration (i.e., not less than 25% based on the total weight of the preparation). In some of the compositions of the invention, the combination cannabinoids/phospholipids is approximately equally proportioned, at a weight ratio in the range from 4:3 to 3:4 (that is, 1:0.75-1.33). We have also found that cannabinoids are absorbed efficiently from the non-solid pharmaceutical preparations that are based on such cannabinoids/phospholipids combinations (i.e., liquids, viscous liquids, gel-like preparations), through the nasal, rectal and vaginal cavities. That is, the compositions of the invention are suitable for intranasal, intrarectal and intravaginal administration, enabling cannabinoids to reach the brain/central nerve system (via nasal administration) or exert a systemic effect (via nasal, rectal or vaginal route of administration).

Accordingly, the invention is primarily directed to a composition for administration into a body cavity selected from the group consisting of nasal cavity, rectal cavity and vaginal cavity, wherein the composition is an essentially non-aqueous composition comprising:

not less than 25% by weight of one or more cannabinoid(s); and from 25% to 55% by weight one or more phospholipid(s).

The composition preferably comprises one or more antioxidant(s).

The term “non-aqueous”, as used herein, refers to compositions that are essentially water-free (i.e., containing less than 10 wt %, less than 5 wt %, less than 1 wt %, less than 0.5 wt % water, in particular water-free (0% water)). Compositions comprising from 25 to 70%, e.g., from 25 to 55%, more specifically 30% to 50% by weight of one or more cannabinoid(s) and from 30% to 50% by weight of one or more phospholipid(s) are preferred. Concentrations reported herein are by weight percentage based on the total weight of the composition, unless indicated otherwise.

As pointed out above, some compositions of the invention are based on roughly equally proportioned mixtures of cannabinoid(s) to phospholipid(s). By “roughly equally proportioned mixtures” are meant mixtures where the weight ratio cannabinoid(s) to phospholipid(s) is in the range from 4:3 to 3:4 (1:0.75-1.33), e.g., from 5:4 to 4:5 (1:0.8-1.25), for example about 1:1.

The compositions of the invention are suitable for use absent added liquid. However, with the aid of liquids such as glycols and oils to be added to cannabinoids/phospholipids-based pharmaceutical preparations, the fluidity and consistency of the preparation can be adjusted to suit the intended use, i.e., intended route of administration (intranasal, intrarectal or intravaginal). In general, the compositions of the invention are in the form of a liquid, a viscous liquid or they have a jelly-like consistency. For example, compositions of the invention could benefit from the addition of increased amount of liquid, say, not less than 15%, or not less than 18%, up to 40% by weight.

The cannabinoid compounds, either natural or synthetic, may be utilized in a solid form (for example, an isolated synthetic compound that underwent purification by crystallization), or in the form of an extraction concentrate, solvent extract, oil extract and oil solution, possibly surfactant-containing extracts and solutions. The concentration of the cannabinoid(s) in the intranasally, intrarectally and intravaginally administrable compositions of the invention may be as high as 50%-70% by weight, e.g., from 30 to 45% by weight, based on the total weight of the composition. A non-limiting list of cannabinoids is given below:

CBD (chemical named 2-[3-methyl-6-(1-methylethenyl)-2-cyclohexen-1-yl]-5-pentyl-1,3-benzenedi-ol). The synthesis of CBD was described, for example, by Gaoni Y, Mechoulam R [Tetrahedron Letters. 26 (8): 1083-1086 (1985)]; and by Petilka et al. [Helv. Chim. Acta, 52:1102 (1969); and in J. Am. Chem. Soc., 87:3273 (1965)].

Δ⁹-THC, available under the name dronabinol; and Δ⁸-THC.

CBN (chemically named 6,6,9-trimethyl-3-pentyl-6H-dibenzo[b,d]pyran-1-ol). The synthesis of CBN was described by Novak et al., Tetrahedron Letters, 23:253 (1982); and by Jesse A. Teske and Alexander Deiters Org. Lett., 2008, 10 (11), pp 2195-2198.

Nabilone (chemically named: 3-(1,1-dimethylheptyl)-6,6a,7,8,10,10a-hexahydro-1-hydroxy-6,6-dimethyl-9-H-dibenzo[b,d]pyran-9-one). The preparation of this synthetic cannabinoid is described, for example, in U.S. Pat. No. 3,968,125.

Levonantradol (chemically named: (−)-(6S,6aR,9R,10aR)-5,6,6a,7,8,9,10,10a-octahydro-6-methyl-3-[(R)-1-meth-yl-4-phenylbutoxy]-1,9-phenanthridinediol 1-acetate. The preparation of this synthetic cannabinoid is described, for example, in U.S. Pat. Nos. 4,206,225, 4,232,018, 4,260,764, 4,235,913, 4,243,674, 4,263,438, 4,270,005, and 4,283,569.

(−)-HU-210 (chemically named: (−)-(3S,4S)-7-hydroxy-Δ⁶-tetrahydrocannabinol-1,1-dimethylhept-yl). The preparation of this synthetic cannabinoid can be found in U.S. Pat. Nos. 4,876,276 and 5,521,215.

(+)-HU-210 (chemically named: (+)-(3S,4S)-7-hydroxy-Δ⁶-tetrahydrocannabinol-1,1-dimethylhept-yl). The preparation of this synthetic cannabinoid is described in U.S. Pat. Nos. 4,876,276 and 5,521,215.

11-hydroxy-Δ⁹-THC, which can be prepared via the synthetic route described by Siegel et al., J. Org. Chem., 54:5428 (1989).

Δ⁸-tetrahydrocannabinol-11-oic acid, which is naturally occurring derivative and can be produced synthetically employing methods described in U.S. Pat. No. 6,162,829.

CP 55,940 (chemically named: 4-(1,1-dimethylheptyl)-2,3′ dihydroxy-6′alpha-(3-hydroxypropyl)-1′,2′,3′,4′,5′,6′-hexahydrobiphenyl), which is commercially available from Tocris Cookson, Inc., Its preparation has been described; see for example U.S. Pat. Nos. 4,371,720 and 4,663,474.

R(+)-WIN 55,212-2 (chemically named: (R)-(+)-[2,3-dihydro-5-methyl-3-(4-morpholinylmethyl)-pyrrolo[1,2,3-de]-1-,4-benzoxazin-6-yl]-1-naphthalenyl-methanone) is commercially available in the form of its mesylate salt from various manufacturers.

It should be noted that the compounds listed above may be used in the form of pharmaceutically acceptable salts or metabolic precursors (e.g., prodrugs that are metabolized in the patient's body as described in U.S. Pat. No. 5,847,128) and their oily solutions. Crude herbal cannabis—in countries and jurisdictions where it is, or will become, legally allowed—can also be delivered using the composition of this invention.

The preferred cannabinoids are selected from the group consisting of CBD, THC, CBN, and mixtures thereof.

Turning now to the phospholipids, they are present in the compositions of the invention at a concentration in the range from 25 to 55%, preferably from 30 to 50% by weight based on the total weight of the composition, more specifically from 30 to 45% by weight, e.g., from 30 to 40%. Phospholipids suitable for use in the preparation of the composition according to the present invention include phosphoglycerides, e.g., phosphatidylcholine (lecithin, such as soy and egg lecithin). Other phospholipids can be selected from hydrogenated phosphatidylcholine, phosphatidylserine, phosphatidylethanolamine, phosphatidylglycerol, phosphatidylinositol and mixtures thereof. Suitable phosphatidylcholine products are commercially available from various sources, for example, from Lipoid under the brand names of Phospholipon®: the 85 G, 90G and 80H, 90H grades and their mixtures; Lipoid®: Lipoid 100S PC, Lipoid S 100, Lipoid S 75, their mixtures and others.

Antioxidants are present in the compositions of the invention, e.g., at a concentration from 0.05 to 1.5% by weight based on the total weight of the composition. Suitable antioxidants include tocopherols and tocopherol derivatives (vitamin E), 3,5-Di-Cert-4-butylhydroxytoluene (BHT), butylated hydroxyanizole (BHA), vitamin C, sodium metabisulfite, potassium metabisulfite, ascorbic acid, lycopene, ascorbyl palmitate and the like. Mixtures of antioxidants may be used.

It may be appreciated that the nasally, rectally and vaginally administrable compositions of the invention share the same basic carrier consisting of the non-aqueous, non-solid cannabinoids/phospholipids system, but each of the compositions may be adapted for its intended use and therefore possesses unique characteristics. We now describe examples of the nasally, rectally and vaginally administrable compositions and their methods of administration, in detail.

One aspect of the invention relates to compositions with high cannabinoid content, suitable for intranasal delivery to the brain, central nerve system (CNS) and/or for systemic administration through the circulation. In its most general form, the composition of the invention is prepared by mixing or more cannabinoid(s) with one or more phospholipids, adding one or more antioxidant(s) according to the relative amounts indicated above, and optionally one or more liquids selected from glycols and oils (vegetable oils such as hemp seed oil, olive oil, sesame oil, brassica seed oil and pomegranate oil).

The glycol used is a water-miscible diol such as propylene glycol. The glycol content of the composition is from 1% by weight based on the total weight of the composition, and up to about 40% by weight, more specifically, from 5 to 30% by weight. It should be noted that the composition of the invention is essentially water-free and in general is also devoid of (C2-C4) volatile mono-alcohols such as ethanol and isopropanol which are used in phospholipids-based vesicular preparations. That is, phospholipids are not arranged in a vesicular structure in the composition of the invention. However, small amounts low alcohols can still be present in the composition, for example, each up to 5-10% by weight based on the total weight of the composition, as long as their presence does not cause the phospholipids to take-up a vesicular structure.

In some preferred embodiments of the invention, the total concentration of the liquid component used in the preparation of the composition (the glycol(s), the vegetable oil(s) or a mixture thereof) is not less than 15% by weight, e.g., not less than 18% by weight.

When the oil is added as a sole liquid component of composition, its concentration may be from 18 to 50% by weight. When present in conjunction with glycol(s), the concentration of the vegetable oil in the composition is not less than 0.005% by weight, preferably from 0.02 to 15%, e.g., 0.5 to 10%, for example from 1 to 5% by weight.

Vegetable oils for use in the invention include, but are not limited to, hemp seed oil, sesame oil and olive oil. Hemp seed oil is produced by cold pressing the seeds of the Cannabis sativa and should not be confused with extractable materials made from the cannabis flower and leaves. Hemp seed oil may be used in the present invention either in a crude form (protein-containing) or in a refined form, following removal of the proteins.

It should be noted that certain vegetable oils possess useful therapeutic properties in their own rights and may demonstrate a desired effect once they reach the brain, e.g., slowing down brain degenerative processes, manage anxiety and depression. Such oils include black cumin seed oil, hemp seed oil, pomegranate seed oil, sesame seed oil, brassica seed oil and black sesame oil, to name a few. For example, pomegranate seed oil has been shown to display neuroprotective effect (see Yuan et al. ACS Chem. Neurosci., 2016, 7 (1), pp 26-33).

Hence, the present invention further relates to compositions comprising a combination of one or more cannabinoids, one or more phospholipid(s), an antioxidant and one or more oils possessing health benefits, especially pomegranate seed oil. The combination of the cannabinoid(s) and the therapeutically effective oil is preferably in proportions by weight of 1:2 to 10:1.

For example, one variant of the composition of the invention comprises from 25 to 40% by weight of cannabinoid(s), from 30 to 50% by weight phospholipids; from 10 to 30% of propylene glycol; from 3 to 20% by weight of therapeutically effective oil, and optionally from 0.1 to 1.5 antioxidant.

Additional nasally, rectally, and vaginally administrable compositions disclosed herein comprise at least one cannabinoid, at least one phospholipid, an antioxidant and at least one therapeutically effective vegetable oil, wherein the weight ratio (cannabinoid(s)+therapeutically effective oil):phospholipids is in the range from 1:0.5-1.5. In glycol free-formulations, the combination of the cannabinoid(s) and the therapeutically effective oil is preferably in proportions by weight from 2:1 to 1:2. Such compositions are generally essentially devoid of water or water-miscible components.

Accordingly, another variant of the composition of the invention comprises from 25 to 35% by weight of cannabinoid(s), from 25 to 50% by weight phospholipids (e.g., to 35%); from 18 to 50% by weight of therapeutically effective oil (e.g., from 20 to 50%), and optionally from 0.1 to 1.5 antioxidant.

In addition to the components already listed above, compositions of the invention may further include auxiliary agents, such as surfactants, preservatives, thickening agents, viscosity and absorption enhancing agents, tolerance enhancers to reduce or prevent drying of the mucus membrane and to prevent irritation thereof.

Suitable preservatives that can be used with the composition include preservatives acceptable for nasal use, for example, benzyl benzalkonium salts, such as benzalkonium chloride. A suitable concentration of the preservative will be from 0.02 to 2% based on the total weight, although there may be some variation depending upon the agent selected.

Regarding thickening agents, the viscosity of the composition can be adjusted at a desired level using a pharmaceutically acceptable thickening agent. However, owing to the suitably proportioned cannabinoids/phospholipids combination, the formulations of the invention may be designed to attain gel-like consistency even in the absence of a thickening agent, to remain in contact in the nasal cavity to enable efficient absorption.

The weight ratio between the components in some preferred compositions according to the invention, namely, cannabinoid(s):phospholipids:glycol:antioxidant is from 1:0.75-1.25:0.20-1.2:0.01-0.05. Some illustrative compositions are set out below (% by weight based on the total weight of the composition):
(i) from 25 to 35% by weight of cannabinoid(s), (e.g., from 30 to 35%, for example, of CBD), from 25 to 35% of phospholipids (e.g., from 30 to 35%), from 30 to 40% of propylene glycol and optionally from 0.1 to 1.5 antioxidant such as vitamin E.
(ii) from 35 to 45% by weight of cannabinoid(s) (e.g., CBD), from 35 to 45% of phospholipids, from 10 to 30% of propylene glycol (e.g., 18-30%) and optionally from 0.1 to 1.5 antioxidant such as vitamin E.
(iii) from 25 to 40% by weight of CBD, from 1 to 25% by weight of THC, from 30 to 45% of phospholipids, from 1 to 30% of propylene glycol (e.g., 5-30%) and optionally from 0.1 to 1.5 antioxidant such as vitamin E.
(iv) from 25 to 35% by weight of CBD, from 1 to 15% by weight of THC, from 1 to 15% by weight of CBN, from 30 to 45% of phospholipids, from 1 to 25% of propylene glycol (e.g., 5-25%) and optionally from 0.1 to 1.5% (0.3-1.5%) of antioxidant such as vitamin E.

Turning now to the preparation of the compositions of the invention, different techniques may be employed to produce homogeneous mixtures consisting of the components listed above. The components may be mixed and homogenized consecutively or simultaneously. That is, one of the components may be mixed and homogenized with another, and this procedure is repeated until all components are combined together. One possible order of addition involves first combining the one or more phospholipids and the one or more cannabinoids to obtain a homogeneous mixture, followed by addition of the antioxidant and lastly the liquid component, e.g., glycol, oil or both. On a laboratory scale, when the amount of the mixture is small, the composition may be mixed using, for example, mortar and pestle. On a larger scale homogenization is achieved using an acceptable instrument such as homogenizer or a mixer.

It should be noted that the composition of the invention is not limited to the delivery of cannabinoid as the sole active ingredient, namely, it may be used to provide combination therapy. That is, a second active ingredient could be added to the composition, as discussed below.

The compositions of the invention can be prepared as liquids, viscous liquids or preparation having a gel consistency (at room temperature). It can also be incorporated into different dosage forms acceptable for the relevant routes of administration, e.g., they may be incorporated into various nasal creams, nasal ointments, nasal lotions and nasal gels in addition of course to nasal liquids.

The compositions of the invention are suitable for intranasal, intrarectal and intravaginal administration, enabling cannabinoids to reach the brain/central nerve system (via nasal administration) or exert a systemic effect (via nasal, rectal or vaginal route of administration) or a topical effect (via rectal and vaginal route of administration). We call the compositions of the invention “CANNASAL”.

As used herein, nasally administering or nasal administration includes administering the compositions into nostrils of the nose to the mucous membranes of the nasal passage or nasal cavity of the mammal. For example, the compositions of the invention can be delivered to the nasal cavity as drops; liquid delivered to the nasal cavity as non-aerosol spray (packaged in a bottle with an atomizer attachment, such as a pump-sprayer) or as an aerosol spray packed in a container under pressure to emit pressurized formulation, as described in detail in Remington's Pharmaceutical Sciences (16th edition, Chapters 83 and 92). Suitable devices [nasal sprays, metered-dose sprays, squeeze bottles, liquid droppers, disposable one-dose droppers, nebulizers, cartridge systems with unit-dose ampoules, single-dose pumps, bi-dose pumps, multiple-dose pumps] are of course commercially available from various sources. Regarding spray devices, it should be noted that both single (unit) dose or multiple dose systems may be used. Typically, a spray device comprises a bottle and a pump. The volume of liquid that is dispensed in a single spray actuation is in the range of from 5 to 250 microlitters/each nostril/single administration and the concentration of the active ingredient in the formulation may be readily adjusted such that one or more spray into the nostrils will comply with the dosage regimen. Administration of compositions of the present invention may also take place using a nasal tampon or nasal sponge containing the compositions. Nasal applicators for nasal gels that are available in the market can be used to deliver viscous or gel-like CANNASAL formulations.

The rectally and vaginally administrable compositions of the invention could be devoid of added liquid components. That is, compositions with gel consistency created by the cannabinoids/phospholipids mixture are preferred for these routes of administration.

For example, rectal and vaginal preparations may be composed solely of cannabinoids, phospholipids and the antioxidant. The addition of liquid such as glycol and oil as set out in detail above is possible, but it is preferred to keep the liquid content of the preparation below 10-15% by weight. Hence, the invention includes a composition for rectal and vaginal administration, comprising from 35 to 50% by weight one or more cannabinoids and from 35 to 55% by weight phospholipid(s), optionally with added liquid.

The rectally and vaginally administrable composition of the invention could be packed and delivered from suitable single-dose containers, tube shaped or bellow-shaped bottles. Additionally, vaginal and rectal applicators suitable for delivery of pharmaceutical preparations including gels, cream and lotions are known (for example, U.S. Pat. No. 7,591,808). For example, gel-like composition may be dispended from a pre-filled single unit-dose vaginal applicator, that can be disposed after use. Alternatively, the vagina is contacted with a gel-like pharmaceutical composition of the present invention that is pressed through a tubular applicator from a storage vessel, e.g., squeezable tube, or the like, into the vagina. The volume of gel-like composition stored in the vessel is designed to constitute a single dose, or two or more doses, so that the vessel can be resealed with suitable closure means to enable repeated application.

The experimental results reported below indicate an efficient delivery of Near Infrared (NIR) probe-containing CANNASAL (indocyanine green; abbreviated ICG) via the nasal route into mice brain. Significantly stronger signals were measured for the probe, in comparison with control formulations. Enhanced delivery to the brain via the nasal route using CANNASAL was also measured by multiphoton imaging (fluorescein isothiocyanate (FITC)), in comparison with control nasal formulation.

Experimental results reported below includes also comparison of the analgesic activity of CBD delivered either from rectally administrable composition of the invention or from an oral solution (analgesic activity of the test formulation is indicated by decrease in the frequency of writhes in the animal model). Rapid and prolonged significant analgesic effect was demonstrated by the rectal formulation; very good writhing inhibition was achieved with the aid of the rectally administrable composition of the invention.

Accordingly, cannabinoids can be administered via the nasal, rectal or vaginal routes with the aid of the composition of the invention to treat any disease or condition where cannabinoids could have impact, e.g., by combating the progress of the disease, or by relieving symptoms associated with the disease. The following diseases and conditions that are treated by cannabinoids can be mentioned: neurological disorder, muscular disturbances, ticks, insomnia, pain, anxiety, migraine, glioma, epilepsy, blastoglioma, cancer, acne, IBD, Chron's disease, loss of appetite, anxiety, distress, panic, tremor, multiple sclerosis, menopause including symptoms associated with menopause such as hot flushes, autism, dementia, Alzheimer, Parkinson, awakens, mood disorders, post-trauma, alcoholic and nonalcoholic fatty liver, hysteria, seizure and types of encephalopathy, including hepatic-encephalopathy and other liver diseases such as hepatic cancer and cirrhosis, menstrual pain and cramps, premenstrual pain, painful menstrual periods and vaginal mucosa inflammation, i.e., local vaginal conditions including infections, inflammation, pain, itch and dryness.

Hence, another aspect of the invention is a method of treatment, in particular treatment of illnesses and conditions set out above and/or symptoms associated therewith, which method comprises the administration (e.g., nasally, rectally or vaginally) to a patient of a composition comprising at least one cannabinoid, phospholipids, an antioxidant and optionally glycol, optionally a vegetable oil, as described above.

One specific aspect of the invention is a method for treating (relieving) pain, for example, in patients with neurological diseases, such as multiple sclerosis, or chronic pain (e.g., pain associated with the nervous system), comprising the intranasal administration of CANNASAL, as described above.

Another specific aspect of the invention is a method for treating liver diseases or relieving their symptoms, comprising the intranasal or rectal administration of the composition of the invention, as described above.

As mentioned above, the nasal delivery is to exert systemic effect through the circulation or for brain/CNS administration for curing brain disease. Pharmaceutically active compounds can be added to the composition of the invention, such as analgesics (including opioid analgesics), sedative, anti-anxiety drugs and anticonvulsants, for example, tramadol HCl, diazepam, brotizolam and butarphenol. Additional active agents that could be delivered by means of the composition of the invention are set out in the following non-limiting list:
Antimalarial agents (e.g. artemisinin derivatives, dihydroartemisinin, artemotil, chloroquine, primaquine, doxycillin, quinine, aminoquinolines, cinchona alkaloids, antifolates, quinidine, melfoquine, halofantrine, lumefantrine, amodiaquine, pyronaridine, tafenoquine, artesunates, artemether, artemotil, biguanides, proguanil, chloproguanil, diaminopyrimidines, pyremethamine, trimethoprim, dapsone, sulfonamides, atovaquone, sulfadoxine-pyrimethamine, N-acetyl cysteine, piperaquine, DHA-piperaquine, lumefantrine, dermaseptins, bisphosphonates, quercitin etc. The drugs could be used alone or in combinations.)
OTC drugs (e.g. antipyretics, anesthetics, cough suppressants, etc.)
Antiinfective agents Anti-malaria agents (such as dihydroartemisinin, etc.)
Antibiotics (e.g. penicillins, cephalosporins, macrolids, tetracyclines, aminoglycosides, anti-tuberculosis agents, doxycycline, ciprofloxacine, moxifloxacine, gatifloxacine, carbapenems, azithromycine, clarithromycine, erythromycine, ketolides, penems, tobramyicin, filgrastim, pentamidine, microcidin, clerocidin; amikacine, etc.)
Genetic molecules (e.g. Anti-sense oligonucleotides, nucleic acids, oligonucleotides, DNA, RNA,
Anti-cancer agents (e.g. anti-proliferative agents, anti-vascularization agents, taxol, etopside, cisplatin, etc.)
Anti-protozoal agents
Antivirals (e.g. acyclovir, gancyclovir, ribavirin, anti-HIV agents, anti-hepatitis agents, famciclovir, valaciclovir, didanosine, saquinavir, ritonavir, lamivudine, stavudine, zidovudine, etc.)
Anti-inflammatory drugs (e.g. NSAIDs, steroidal agents, cannabinoids, leukotriene-antagonists, tacrolimus, sirolimus, everolimus, etc.)
Anti-allergic molecules (e.g. antihistamines, fexofenadine)
Bronchodilators
Vaccines and other immunogenic molecules (e.g. tetanus toxoid, reduced diphtheria toxoid, acellular pertussis vaccine, mums vaccine, smallpox vaccine, anti-HIV vaccines, hepatitis vaccines, pneumonia vaccines, influenza vaccines, TNF-alpha-antibodies etc.)
Anesthetics, local anesthetics.
Antipyretics (e.g. paracetamol, ibuprofen, diclofenac, aspirin, etc.)
Agents for treatment of severe events such cardiovascular attacks, seizures, hypoglycemia, etc.
Afrodisiacs from plants or synthetics
Anti-nausea and anti-vomiting.
Immunomodulators (immunoglobulins, etc.)
Cardiovascular drugs (e.g. beta-blockers, alpha-blockers, calcium channel blockers, etc.)
   steroid hormones (eg. insulin, insulin derivatives, insulin detemir, insulin monomeric, oxytocin, LHRH, LHRH analogues, adreno-corticotropic hormone, somatropin, leuprolide, calcitonin, parathyroid hormone, estrogens, testosterone, adrenal corticosteroids, megestrol, progesterone, sex hormones, growth hormones, growth factors, etc.)
Vitamins (e.g. Vit A, Vitamins from B group, folic acid, Vit C, Vit D, Vit E, Vit K, niacin, derivatives of Vit D, etc.)
   Autonomic Nervous System Drugs
Fertilizing agents
Antidepressants (e.g. buspirone, venlafaxine, benzodiazepins, selective serotonin reuptake inhibitors (SSRIs), sertraline, citalopram, tricyclic antidepressants, paroxetine, trazodone, lithium, bupropion, sertraline, fluoxetine, etc.)
Agents for smoking cessation (e.g. bupropion, nicotine, etc.)
Agents for treating alcoholism and alcohol withdrawal
Lipid-lowering agents (eg. inhibitors of 3 hydroxy-3-methylglutaryl-coenzyme A (HMG-CoA) reductase, simvastatin, atrovastatin, etc.)
Drugs for CNS or spinal cord (benzodiazepines, lorazepam, hydromorphone, midazolam, Acetaminophen, 4′-hydroxyacetanilide, barbiturates, anesthetics, etc.)
   Anti-epilepsic agents (e.g. valproic acid and its derivatives, carbamazepin, etc.)
Angiotensin antagonists (e.g. valsartan, etc.)
Anti-psychotic agents and anti-schizophrenic agents (e.g. quetiapine, risperidone)
Agents for treatment of Parkinsonian syndrome (e.g. L-dopa and its derivatives, trihexyphenidyl, etc.)
Anti-Alzheimer drugs (e.g. cholinesterase inhibitors, galantamine, rivastigmine, donepezil, tacrine, memantine, N-methyl D-aspartate (NMDA) antagonists).
Agents for treatment of non-insulin dependent diabetes (e.g. metformine,
Agents against erectile dysfunction (e.g. sildenafil, tadalafil, papaverine, vardenafil, PGE1, etc.)
Prostaglandins
Agents for bladder dysfunction (e.g. oxybutynin, propantheline bromide, trospium, solifenacin succinate etc.)
Agents for treatment menopausal syndrome (e.g estrogens, non-estrogen compounds, etc.)
Agents for treatment hot flashes in postmenopausal women
Agents for treatment primary or secondary hypogonadism (e.g. testosterone, etc.)
Cytokines (e.g. TNF, interferons, IFN-alpha, IFN-beta, interleukins etc.)
CNS stimulants
Muscle relaxants
Anti paralytic gas agents
Appetite stimulators/depressors (e.g. cannabinoids, etc.)
Narcotics and Antagonists (e.g. opiates, oxycodoneetc.)
Painkillers (opiates, endorphins, tramadol, codein, NSAIDs, gabapentine, fentanil and pharmaceutically acceptable salts thereof etc.)
Hypnotics (Zolpidem, benzodiazepins, barbiturates, ramelteon, etc.)
Histamines and Antihistamines
Antimigraine Drugs (e.g. imipramine, propranolol, sumatriptan, eg.)
Diagnostic agents (e.g. Phenolsulfonphthalein, Dye T-1824, Vital Dyes, Potassium Ferrocyanide, Secretin, Pentagastrin, Cerulein, etc.)
Topical decongestants or anti-inflammatory drugs
Anti-acne agents (e.g. retinoic acid derivatives, doxicillin, minocyclin, etc.)
ADHD related medication (e.g. methylphenidate, dexmethylphenidate, dextroamphetamine, d- and 1-amphetamin racemic mixture, pemoline, etc.)
Diuretic agents
Anti-osteoporotic agents (e.g. bisphosphonates, alendronate, pamidronate, tirphostins, etc.)
Drugs for treatment of asthma drugs for post trauma, crisis, anxiety treatment
Anti-spasmotic agents (e.g. papaverine, etc.)
Agents for treatment of multiple sclerosis and other neurodegenerative disorders (e.g. mitoxantrone, glatiramer acetate, interferon beta-la, interferon beta-lb, etc.)
Plant derived agents from leave, root, flower, seed, stem or branches extracts.

Due to the efficiency with which cannabinoids are absorbed from the compositions of the invention, the concentration and amount (e.g., dosage unit) of the formulation may be readily adjusted such that its delivery (e.g., to the nostrils, rectum and vagina)) comply with the selected dosage regimen. A therapeutically effective amount in the methods of treatment provided by the present invention may be from 10 mcg to 1000 mg per kg body weight of the patient treated by the methods described above, per day, e.g., from 10, 25, 50, 75, 100, 150, 200, 300 mcg per kg per day up to 1, 10 100, 500, 600, 700, 800, 900 and 1000 mg/kg/day.

In the drawings:

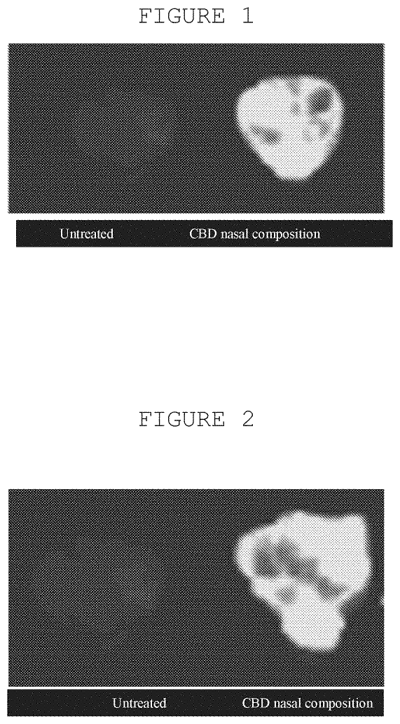

FIG. 1: Representative NIR images for mice brains treated with 10 μl CBD nasal composition (right) as compared to brain of untreated mice (left).

FIG. 2: Representative NIR images for mice brains treated with 10 μl CBD nasal composition (right) as compared to the brain of untreated mice (left).

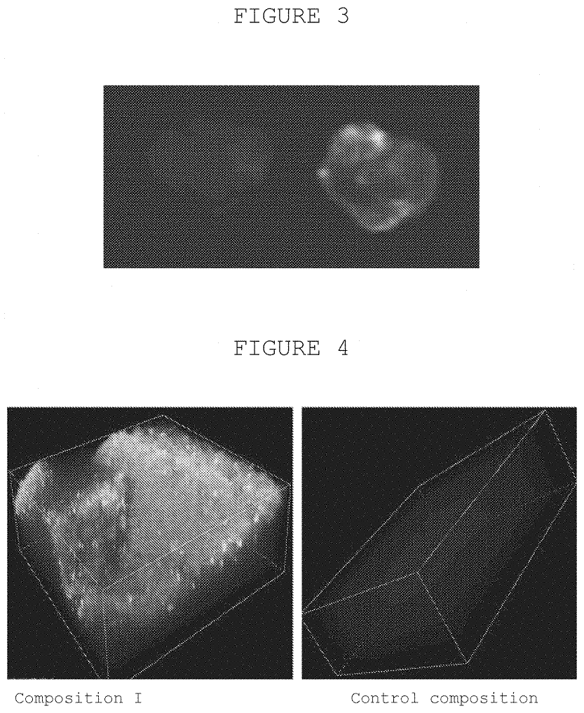

FIG. 3: Representative NIR images for mice brains treated with 10 μl nasal control compositions containing 0.5% w/w ICG (right) as compared to untreated mice (left).

FIG. 4: Representative multiphoton micrographs for the olfactory region in mice brains treated nasally with 10 μl of Composition I or Control Composition, each containing 0.5% w/w FITC. Field of images: height: 818 μm, width: 818 μm and depth: 200 μm; lens ×20 (A1-MP microscope NIKON-Japan).

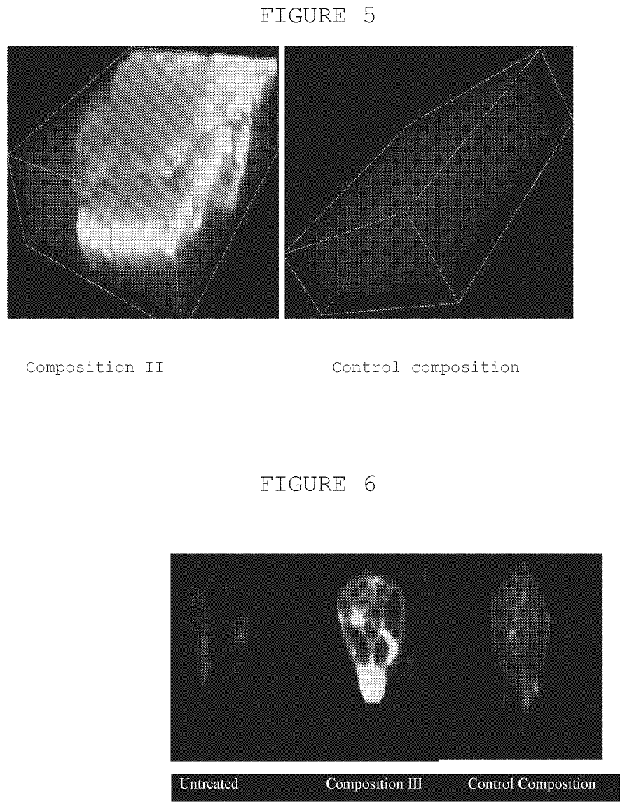

FIG. 5: Representative multiphoton micrographs for the olfactory region in mice brains treated nasally with 10 μl of Composition II or Control Composition, each containing 0.5% w/w FITC. Field of images: 818 μm, width: 818 μm and depth: 200 μm; lens ×20 (A1-MP microscope NIKON-Japan).

FIG. 6: Representative NIR images for mice brains treated nasally with 10 μl of Composition III or Control Composition each containing 0.5% w/w ICG as compared to untreated mice.

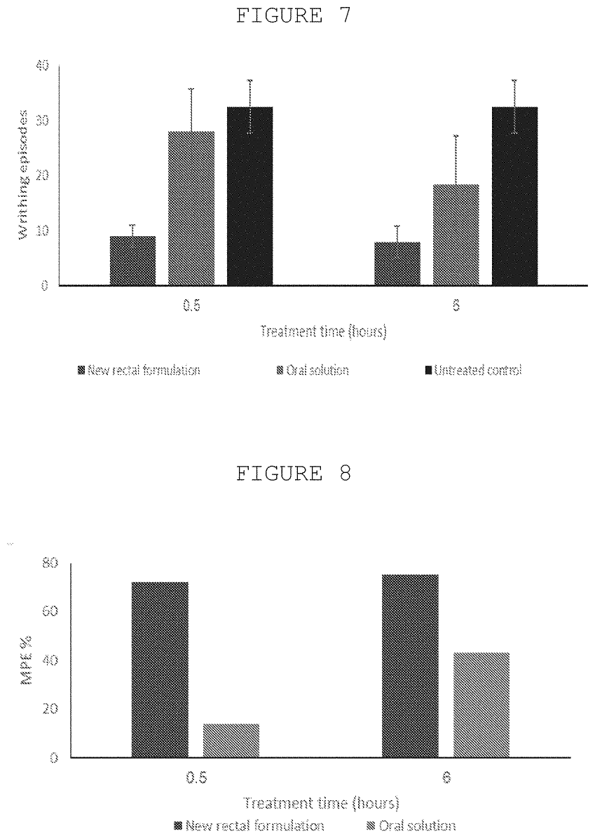

FIG. 7: Mean writing counts in mice treated with 50 mg/kg CBD from the rectal formulation of the invention as compared to oral solution, 0.5 and 6 hours prior to IP injection of acetic acid and compared to untreated control mice received IP injection of acetic acid, n=5 for untreated control, n=3 for the rectal formulation and oral solution at 0.5 hour and n=5 for the two treatments after 6 hours. (Mean±SD). p<0.05 (considered significant) for the rectal formulation vs. untreated control at 0.5 and 6 hours and vs. oral solution at hours. p>0.05 (considered not significant) for oral solution vs. untreated control at 0.5 and 6 hours, by two-tailed Mann-Whitney.

FIG. 8: MPE % values in mice treated with 50 mg/kg CBD from the rectal formulation as compared to oral solution, 0.5 and 6 hours prior to IP injection of acetic acid and compared to untreated control mice received IP injection of acetic acid.

### EXAMPLES

Glossary: PL—phospholipids; PG—propylene glycol; CBD—Cannabidiol; THC—Tetrahydrocannabinol; CBN—Cannabinol; HSO—hemp seed oil; Vit E—vitamin E; Fluorescein isothiocyanate: FITC. CBD obtained by extraction from plants, purity 93.7% (Aifame, Switzerland). THC obtained by extraction from plant, purity>90% (BOL pharma, Israel). Lipoid 5100 and Phospholipon 90 G are from Lipoid GmbH, Germany. Pomegranate Oil (organic) manufactured by Bara Herbs, Israel and Hemp Seed Oil (organic) manufactured by Pukka Herbs, UK were used. Lecithin Soya (Fagron, Spain) and Propylene glycol (Tamar) from Tamar, Israel. Olive Oil from Henry Lamotte Oil GmbH, Germany.

### Example 1

### CBD-Containing Composition

TABLE 1

| Ingredients | Concentration % w/w |
|---|---|
| CBD | 41.3 |
| Phospholipon ® 90G | 41.3 |
| Vitamin E | 1.3 |
| Propylene glycol | 16.1 |

Preparation: PL was mixed well, then CBD was added and mixed well. Vitamin E was added and mixed. Finally, PG was added with mixing. A viscous liquid was obtained.

### Example 2

### CBD-Containing Composition

TABLE 2

| Ingredients | Concentration % w/w |
|---|---|
| CBD | 39.3 |
| Phospholipon ® 90G | 46.8 |
| Vitamin E | 1.4 |
| Hemp seed oil | 4.7 |
| Propylene glycol | 7.8 |

Preparation: PL was mixed well then CBD was added and mixed well. Then Vit E was added, followed by the addition of HSO with mixing. Finally, PG was added and mixed. A viscous liquid was obtained.

### Example 3

### CBD-Containing Composition

TABLE 3

| Ingredients | Concentration % w/w |
|---|---|
| CBD | 42.1 |
| Phospholipon ® 90G | 42.1 |
| Vitamin E | 1.5 |
| Hemp seed oil | 4.3 |
| menthol | 0.2 |
| Propylene glycol | 9.8 |

Preparation: PL was mixed well then CBD was added and mixed well. Then Vitamin E was added followed by addition of HSO with mixing. Then menthol was added and mixed well. Finally, PG was added with mixing. A viscous liquid was obtained.

### Example 4

### CBD-Containing Composition

TABLE 4

| Ingredients | Concentration % w/w |
|---|---|
| CBD | 37.8 |
| Phospholipon ® 90H | 37.8 |
| Vitamin E | 1.3 |
| Olive oil | 9.5 |
| Propylene glycol | 13.6 |

Preparation: Phospholipon® 90H was mixed well then CBD was added and mixed well. Then Vitamin E was added, followed by olive oil addition with mixing. Finally, PG was added with mixing. A viscous liquid was obtained.

### Example 5

### CBD-Containing Composition

TABLE 5

| Ingredients | Concentration % w/w |
|---|---|
| CBD | 32 |
| Phospholipon ® 90G | 32 |
| Vitamin E | 0.5 |
| Propylene glycol | 35.5 |

Preparation: PL was mixed well then CBD was added and mixed well. Then Vit E is added and mixed. Finally, PG is added and mixed. A liquid was obtained.

### Example 6

### CBD-Containing Composition

TABLE 6

| Ingredients | Concentration % w/w |
|---|---|
| CBD | 40 |
| Phospholipon ® 90G | 40 |
| Vitamin E | 0.8 |
| Propylene glycol | 19.2 |

Preparation: PL is mixed well then CBD was added and mixed well. Then Vit E is added with mixing. Finally, PG is added and mixed. A viscous liquid was obtained.

### Example 7

### CBD-Containing Composition

TABLE 7

| Ingredients | Concentration % w/w |
|---|---|
| CBD | 45 |
| Phospholipon ® 90G | 37.8 |
| Vitamin E | 1.3 |
| Olive oil | 4 |
| Propylene glycol | 11.9 |

Preparation: PL was mixed well then CBD was added and mixed well. Then Vit E was added followed by olive oil addition with mixing. Finally, PG was added and mixed.

### Example 8

### CBD-Containing Composition

TABLE 8

| Ingredients | Concentration % w/w |
|---|---|
| CBD | 40 |
| Lipoid S | 40 |
| Vitamin E | 0.8 |
| Propylene glycol | 19.2 |

Preparation: Lipoid was mixed well, then CBD was added and mixed well. Then Vitamin E was added and mixed. Finally, PG was added and mixed. A viscous liquid was obtained.

### Example 9

### Cannabinoid Mixture (CBD+THC)-Containing Composition

TABLE 9

| Ingredients | Concentration % w/w |
|---|---|
| CBD | 30 |
| THC | 10 |
| Lipoid S | 40 |
| Vitamin E | 0.8 |
| Propylene glycol | 19.2 |

Preparation: Lipoid was mixed well with CBD and THC. Then Vitamin E was added with mixing. Finally, PG was added and mixed.

### Example 10

### Cannabinoid Mixture (CBD+THC)-Containing Composition

TABLE 10

| Ingredients | Concentration % w/w |
|---|---|
| CBD | 35 |
| THC | 5 |
| Lipoid S | 40 |
| Vitamin E | 0.8 |
| Propylene glycol | 19.2 |

Preparation: Lipoid was mixed well with CBD and THC. Then Vitamin E was added with mixing. Finally, PG was added and mixed. A viscous liquid was obtained.

### Example 11

### Cannabinoid Mixture (CBD+THC)-Containing Composition

TABLE 11

| Ingredients | Concentration % w/w |
|---|---|
| CBD | 39 |
| THC | 1 |
| Lipoid S | 40 |
| Vitamin E | 1 |
| Propylene glycol | 19 |

Preparation: Lipoid was mixed well with CBD and THC. Vitamin E was added with mixing. Finally, PG was added and mixed.

### Example 12

### Cannabinoid Mixture (CBD+THC)-Containing Composition

TABLE 12

| Ingredients | Concentration % w/w |
|---|---|
| CBD | 30 |
| THC | 20 |
| Phospholipon G | 40 |
| Vitamin E | 0.8 |
| Propylene glycol | 9.2 |

Preparation: Phospholipon G was mixed well with CBD and THC. Vitamin E was added with mixing. Finally, PG was added and mixed. A liquid was obtained.

### Example 13

### Cannabinoid Mixture (CBD+THC)-Containing Composition

TABLE 13

| Ingredients | Concentration % w/w |
|---|---|
| CBD | 30 |
| THC | 10 |
| Lipoid S | 10 |
| Phospholipon ® G | 20 |
| Vitamin E | 0.8 |
| Propylene glycol | 29.2 |

Preparation: Lipoid and Phospholipon® G are mixed well with CBD and THC. Then Vitamin E was added with mixing. Finally, PG was added and mixed. A liquid was obtained.

### Example 14

### Cannabinoid Mixture (CBD+THC+CBN)-Containing Composition

TABLE 14

| Ingredients | Concentration % w/w |
|---|---|
| CBD | 30 |
| THC | 5 |
| CBN | 10 |
| Phospholipon G | 40 |
| Vitamin E | 0.8 |
| Propylene glycol | 14.2 |

Preparation: Phospholipon® G is mixed well with the mixture of drugs (CBD, THC and CBN). Then Vitamin E was added with mixing. Finally, PG was added and mixed.

### Examples 15 to 19

### Composition of CBD with a Second Pharmaceutically Active Compound

TABLE 15

| | Exam- | Exam- | Exam- | Exam- | Exam- |
|---|---|---|---|---|---|
| | ple 15 | ple 16 | ple 17 | ple 18 | ple 19 |
| Ingredient | wt % | wt % | wt % | wt % | wt % |
| CBD | 40 | 30 | 30 | 30 | 30 |
| Tramadol HCl | 1 | — | — | — | — |
| Diazepam | — | 1 | 1 | — | — |
| Brotizolam | — | — | — | 0.25 | — |
| Butarphenol | | | | | 0.5 |
| Phospholipon ® | 35 | 25 | — | 30 | 30 |
| 90G | | | | | |
| Lipoid PC | — | 7 | 15 | — | 5 |
| Phospholipon ® | — | — | 15 | — | — |
| 90H | | | | | |
| Vitamin E | 1 | q.s. | q.s. | 0.75 | 0.5 |
| Propylene | 23 | to 100 | to 100 | 39 | 34 |
| glycol | | | | | |

The compositions of Examples 15 to 19 set out in Table 15 were prepared using the procedures described in previous examples. Phospholipid(s) are combined with the cannabinoids and mixed well, followed by addition of the other drug with mixing, and addition of the antioxidant and PG under mixing.

### Example 20

### Nasal Delivery to Brain Tested by Near Infrared (NIR) Imaging

The delivery of the NIR probe Indocyanin green (ICG) to the cortex in mice brain from CBD nasal composition of the invention was examined by Odyssey® Infrared Imaging System (LI-COR, USA). The composition is tabulated in Table 16.

TABLE 16

| Ingredients | Concentration % w/w |
|---|---|
| ICG | 0.5 |
| CBD | 30 |
| Phospholipon G | 30 |
| Vitamin E | 0.5 |
| Propylene glycol | 39 |

Preparation: CBD was mixed well with PL and then mixed at intervals of ten minutes over a period of half an hour. Then Vitamin E was added followed by addition of ICG. Finally, PG was added and mixed well. A liquid was obtained.

Mice were treated with 10 μl of the above nasal composition. Thirty minutes after treatment, the animals were sacrificed; brains were removed, washed with normal saline and observed under the imaging system. The scanning was performed using offset 2, resolution 339.6 μm, channel 800 nm and intensity 1. The fluorescence intensity of the probe (arbitrary units A.U.) in brain was further assessed using ImageJ software. The results were compared to the brain of untreated mice.

The NIR images obtained in this experiment (FIG. 1) show that the administration of CBD nasal composition containing ICG yielded a strong fluorescent signal as compared to the brain of untreated mice.

Semi-quantification of the images and normalization of the fluorescence intensity (by subtracting the auto fluorescence of the untreated brain from the fluorescence intensity of each image) was carried out. A fluorescence of 16.6 A.U. was assessed in the brain of animals treated with the nasal composition of the invention.

### Example 21

### Nasal Delivery to Brain Tested by Near Infrared (NIR) Imaging

The delivery of the NIR probe Indocyanin green (ICG) to the cortex in mice brain from CBD nasal composition of the invention was examined by Odyssey® Infrared Imaging System (LI-COR, USA). The composition is tabulated in Table 17.

TABLE 17

| Ingredients | Concentration % w/w |
|---|---|
| ICG | 0.5 |
| CBD | 40 |
| Lipoid SPC | 40 |
| Vitamin E | 0.5 |
| Propylene glycol | 19 |

Preparation: CBD was mixed well with Lipoid SPC for five minutes; it was then mixed every ten minutes over a period of additional forty min. Then Vitamin E was added and mixed, then ICG was added and mixed. Finally, PG was added and mixed well. A viscous liquid was obtained.

Mice were treated with 10 μl nasal composition presented in Table 17. Thirty minutes after treatment, the animals were sacrificed; brains were removed, washed with normal saline and observed under the imaging system. The scanning was performed using offset 2, resolution 339.6 μm, channel 800 nm and intensity 1. The fluorescence intensity (arbitrary units A.U.) in brain was further assessed using ImageJ software.

The NIR images obtained in this experiment (FIG. 2) show that the administration of nasal composition of the invention yielded a strong fluorescence in the brain. A fluorescence of 20.4 A.U. was assessed in the group treated with nasal composition of this example.

### Example 22 (Comparative)

### Nasal Delivery to Brain Tested by Near Infrared (NIR) Imaging

Mice were divided into two control treatment groups and one untreated control group.

TABLE 18

| Ingredients | Concentration % w/w |
|---|---|
| ICG | 0.5 |
| Propylene glycol | To 100 |

Preparation: ICG was dissolved in PG with mixing.

Mice were treated nasally with 10 μl of the control composition tabulated in Table 18. Thirty minutes after treatments, the animals were sacrificed; brains were removed, washed with normal saline and observed under the imaging system. The scanning was performed using offset 2, resolution 339.6 μm, channel 800 nm and intensity L1. The fluorescence intensity of the probe (arbitrary units A.U.) in brain was further assessed using ImageJ software.

The NIR images obtained in this experiment (FIG. 3) show that the administration of control nasal compositions containing ICG resulted in only a very weak fluorescent signal as compared to the nasal compositions of the invention presented above.

Semi-quantification of the images and normalization of the fluorescence intensity (by subtracting the auto fluorescence of the untreated brain from the fluorescence intensity of each image) show a fluorescence of only 4.8 A.U. for the control nasal composition.

### Examples 23-25

### Cannabinoid(s) and Oil-Containing Compositions

This set of examples illustrate the incorporation of pomegranate oil into CANNASL formulations; the total concentrations of the cannabinoid(s) and the oil in the illustrated formulations is from ˜36% to 45% by weight.

TABLE 19

| | 23 | 24 | 25 |
|---|---|---|---|
| Ingredient | wt % | | |
| Lipoid S100 | 50.0 | 40.0 | |
| Phospholipon ® 90G | | | 35.0 |
| CBD | 25.0 | 29.0 | 30.0 |
| THC | | 1.0 | |
| CBN | | | 1.0 |
| Pomegranate oil | 12.5 | 15.0 | 6.0 |
| Propylene glycol | 12.5 | 15.0 | 27.0 |

The compositions set out in Table 19 were prepared by the procedures described above. In Examples 23 and 24, propylene glycol was the last added ingredient: it was slowly added under stirring to the phospholipids, cannabinoid(s) and oil mixture. In Example 25, the order of addition was different: Phospholipon® 90G was mixed with the CBD and CBN, followed by the addition of propylene glycol; the pomegranate oil was then added slowly under mixing. In all three cases, homogenous (brownish or yellowish) viscous liquid is obtained.

### Examples 26-31

### Cannabinoid(s) and Oil(s)-Containing Compositions

This set of Examples illustrate the incorporation of cannabinoid (either a single cannabinoid or a mixture of two cannabinoids) and therapeutically effective oils into high content phospholipids preparations.

TABLE 20

| | 26 | 27 | 28 | 29 | 30 | 31 |
|---|---|---|---|---|---|---|
| | % w/w | % w/w | % w/w | % w/w | % w/w | % w/w |
| Lipoid S100 | | 50.0 | | | | 40.0 |
| Lecithin soya | 33.3 | | 40 | 44 | 40 | |
| CBD | 29.0 | 29.0 | 20 | 30 | 30 | 30 |
| THC | 1.0 | 1.5 | 10 | | | |
| Pomegranate | 36.7 | 18.5 | 15 | 10 | 15 | 15 |
| oil | | | | | | |
| Black sesame | | | | | | 10 |
| seed oil | | | | | | |
| Olive oil | | | 15 | 16 | | |
| Hemp seed | | | | | 15 | 5 |
| oil | | | | | | |

The compositions are prepared by first mixing the phospholipids component with the cannabinoid(s), followed by slow or portion wise addition of the oil(s) under mixing. When a mixture of oils is used, such as in Examples 28 to 31, the oils are added either successively (e.g., a portion of one oil is mixed with the phospholipids/cannabinoids, followed by slow addition under mixing of the remaining portion and the other oil, such as in Example 28, or by successive addition of the oils to the phospholipids/cannabinoids with mixing (such as in Examples 29 to 31). Homogeneous brownish liquids or viscous liquids were formed.

### Examples 32-33 (of the Invention) and 34 (Comparative) Nasal Delivery to the Brain of Cannabinoid and Oils-Containing Compositions Measured by Multiphoton Imaging

The compositions tabulated in Table 21 were prepared and then nasally administered to mice; their delivery to the brain via the nasal route was measured by multiphoton imaging.

TABLE 21

| | Example 32 | Example 33 | Example 34 |
|---|---|---|---|
| | Composition I | Composition II | (control) |
| | % w/w | % w/w | % w/w |
| Lipoid SPC | 25.0 | | |
| Lecithin soya | | 30.0 | |
| Pomegranate oil | 12.5 | 15.0 | 20.0 |
| Sesame oil | 36.5 | 24.0 | 49.0 |
| CBD | 25.0 | 30.0 | |
| α-tocopherol | 0.5 | 0.5 | 0.5 |
| FITC | 0.5 | 0.5 | 0.5 |
| Vaseline | | | 30.0 |

Preparation

Composition I (of Example 32) was prepared in the following way. Lipoid SPC was mixed with CBD, followed by addition of α-Tocopherol. Pomegranate oil was added and the mixture was mixed. Then the sesame oil was added slowly under mixing. Lastly, FITC was added under mixing. A liquid was obtained. Composition II (of Example 33) was prepared in the following way. Lecithin was mixed with pomegranate oil, followed by addition of α-Tocopherol. Then sesame oil was added gradually under mixing. CBD was added gradually and slowly under mixing. Lastly, FITC was added under mixing. A liquid was obtained. The Control Composition (of Example 34) was prepared in the following way. Vaseline was mixed with pomegranate oil, followed by addition of α-Tocopherol. Sesame oil was then added gradually under mixing. Lastly, FITC was added and mixed well.

### Experimental Protocol

Mice were treated with 10 μl of Nasal Composition I, II or Control Composition each containing 0.5% w/w FITC. Ten minutes after treatment, the animals were sacrificed; brains were removed, washed with normal saline and the olfactory region in brain was observed under the multiphoton microscope A1-MP microscope (NIKON, Japan). The field of image was 818×818×200 nm (width×height×depth), the scanning was performed using objective lens ×20, excitation wavelength of 740 nm, laser intensity 6%, scan speed 0.125. The fluorescence intensity of the probe (Arbitrary units, A.U.) in scanned brain region was further analyzed using ImageJ software. The brain of untreated mouse was examined to rule out the auto-fluorescence of the olfactory region.

Results

The multiphoton micrographs obtained in this experiment are given in FIGS. 4-5. The results show that the nasal administration of Compositions I and II (containing FITC) yielded a strong fluorescent signal as compared to Control FITC Composition, with highest signal following probe administration from Composition II (FIG. 5). Semi-quantification of the images shows a fluorescent intensity of 63.7 and 77.9 for Composition I and II, respectively. On the other hand, nasal administration of FITC from the Control Composition yielded a fluorescent intensity of only 9.4 A.U.

### Examples 35 (of the Invention) and 36 (Comparative)

### Nasal Delivery to the Brain of Cannabinoid and Oils-Containing Compositions Measured by NIR Imaging

The compositions tabulated in Table 22 were prepared and then nasally administered to mice. The delivery of the NIR probe Indocyanin green (ICG) to the cortex in mice brain from nasal compositions of the invention was examined by Odyssey® Infrared Imaging System (LI-COR, USA).

TABLE 22

| | Example 35 | |
|---|---|---|
| | Composition III | Example 36 |
| | % w/w | (Control) |
| Lipoid SPC | 40.0 | |
| Pomegranate oil | 5.0 | |
| CBD | 35.5 | |
| Propylene glycol | 18.5 | 99.5 |
| α-tocopherol | 0.5 | |
| ICG | 0.5 | 0.5 |

Composition III (of Example 35) was prepared in the following way. Lipoid SPC was mixed with CBD intermittently over approximately one hour, followed by addition of α-Tocopherol. Then pomegranate oil was added and mixed well, followed by slow addition of propylene glycol under mixing. Lastly, ICG was added and mixed well.

The Control Composition (of Example 36) was prepared by dissolving the ICG in propylene glycol.

Experimental Protocol

Mice were treated with 10 μl of Nasal Composition III or Control Composition each containing 0.5% w/w ICG as compared to untreated mice. Thirty minutes after treatments, the animals were sacrificed; brains were removed, washed with normal saline and observed under the imaging system. The scanning was performed using offset 2, resolution 339.6 μm, channel 800 nm and intensity 1. The fluorescence intensity of the probe (Arbitrary units, A.U.) in brain was further analyzed using ImageJ software.

Results

The NIR images obtained in this experiment show that the nasal administration of Composition III containing ICG yielded a strong fluorescent signal as compared to Control ICG Composition (FIG. 6). Semi-quantification of the images and normalization of the fluorescence intensity (by subtracting the auto fluorescence of the untreated brain from the fluorescence intensity of each image) show a fluorescent intensity of 9.7 for Composition III. On the other hand, nasal administration of the Control ICG Composition yielded a fluorescent intensity of only 0.4 A.U.

### Examples 37 (of the Invention) and 38 (Comparative)

### Antinociceptive Effect of Rectal Administration of CBD from the Composition of the Invention

The purpose of the study reported herein was to evaluate the onset and the prolonged antinociceptive effect of cannabidiol (CBD) administered rectally from a formulation of the invention, in comparison with oral solution, in mice model of pain. The two tested formulations are set out in Table 23.

TABLE 23

| | Example 37 | Example 38 |
|---|---|---|
| | Composition III | (comparative - oral solution) |
| | % w/w | % w/w |
| CBD | 40.0 | 20.0 |
| Phospholipon 90 G | 50.0 | |
| Vit E | 10.0 | |
| Propylene glycol | | 40.0 |
| Ethanol absolute | | 40.0 |

Experimental Protocol

This experiment was performed on twenty five male CD-1 ICR mice (21-25 g). Mice were housed under standard conditions of light and temperature in plastic cages in the specific-pathogen unit (SPF) of the pharmacy school at the Hebrew University. Animals were provided with unlimited access to water and food, and were individually inserted in separated cages with smooth flat floor.

Twenty mice were divided randomly and equally into four treatment groups to test the rectal and oral treatments at two different time points; the other five mice served for the control (untreated) group. Animals were treated with CBD at a dose of 50 mg/kg in ˜6-6.5 mg from the formulation of the invention (Example 37) or the oral solution (Example 38). The treatments were administered using Microman® (a precision microliter pipette).

30 minutes or 6 hours after treatments, the animals were injected intraperitoneally with acetic acid (0.6% v/v) at a dose of (10 ml/kg) (n=5). Five animals were anesthetized with Isoflurane® and injected with acetic acid at the same dose without treatment served as untreated control.

Number of writhing episodes was recorded by counting the number of writhes 5 minutes after acetic acid administration for a period of 20 minutes. Writhes were indicated by the abdominal constriction and stretching of at least one hind limb.

The analgesic effect of each treatment is expressed by the Maximum Possible Effect (MPE %) of the treatments, which is directly related to the efficiency of the treatment, and is calculated according to the following equation:

MPE %=[Mean writhing in untreated control group−number of writhing in treated group]/[Mean writhing in untreated control group]

Results

The results of the experiment (onset and prolonged antinociceptive effect of CBD administered in the formulation of the invention as compared to oral solution) are shown in Tables 24 and 25 and are also presented graphically in the form of bar diagrams appended in FIGS. 7 and 8.

Table 24 shows the number of writhes counted after IP injection of acetic acid, at the two time points (Mean±SD at the time points 0.5 h and 6 h). The bars in the diagram of FIG. 7 correspond (from left to right) to the rectal treatment, oral treatment and untreated control.

TABLE 24

Writhes Count

| Time point (hours) | 0.5 | 6 |
|---|---|---|
| New rectal formulation | 9.0 ± 2.0 | 8.0 ± 2.8 |
| Oral solution | 25.0 ± 7.8 | 18.4 ± 8.8 |
| Untreated control | 32.5 ± 4.9 | |

The MPE % values calculated are shown in Table 25 and in FIG. 8 (the left bar represents the rectal treatment and the right bar the oral treatment).

TABLE 25

MPE %

| Time point (hours) | 0.5 | 6 |
|---|---|---|
| rectal formulation | 72.3 | 75.4 |
| Oral solution | 13.8 | 43.4 |

The results indicate that CBD administration to mice male CD-1 ICR mice from the new formulation of the invention lead to rapid and prolonged significant analgesic effect starting from 0.5 hour with 72.3% MPE and reaching to 6 hours with MPE % value of 75.4%. On the other hand, administration of equal dose of CBD from oral solution lead to minimal analgesic effect after 0.5 hour with 13.8% MPE. This effect was increased after 6 hours to 43.4%.

1. A composition for administration into a body cavity selected from the group consisting of the nasal cavity, the rectal cavity and the vaginal cavity, wherein the composition is an essentially non-aqueous composition comprising:
not less than 25% by weight of one or more cannabinoid(s);
from 25% to 55% by weight one or more phospholipid(s); and optionally one or more antioxidants.

2. A composition according to claim 1, comprising:
from 30% to 50% by weight of one or more cannabinoid(s) and from 30% to 50% by weight of one or more phospholipid(s).

3. A composition according to claim 1, wherein the weight ratio cannabinoid(s) to phospholipid(s) is in the range of 1:0.75-1.33.

4. A composition according to claim 3, wherein the weight ratio cannabinoid(s) to phospholipid(s) is in the range of 1:0.8-1.25.

5. A composition according to claim 1, further comprising one or more liquids selected from the group consisting of glycols and vegetable oils.

6. A composition according to claim 1, which is devoid of added liquids.

7. A composition according to claim 1 for nasal administration.

8. A composition according to claim 1 for rectal administration.

9. A composition according to claim 1 for vaginal administration.

10. A composition for nasal administration according to claim 7, comprising not less than 18% by weight of one or more liquids selected from the group consisting of glycols and vegetable oils.

11. A composition for nasal administration according to claim 10, comprising from 25 to 35% by weight of cannabinoid(s), from 25 to 35% of phospholipid(s), from 30 to 40% of propylene glycol and an antioxidant.

12. A composition for nasal administration according to claim 10, comprising from 35 to 45% by weight of cannabinoid(s), from 35 to 45% of phospholipid(s), from 18 to 30% of propylene glycol and an antioxidant.

13. A composition according to claim 7, comprising a vegetable oil selected from the group consisting of black cumin seed oil, hemp seed oil, pomegranate seed oil, sesame seed oil, brassica seed oil and black sesame oil.

14. A composition according to claim 13, comprising pomegranate oil, sesame oil or a mixture thereof.

15. A composition according to claim 13 for nasal administration, comprising from 25 to 35% by weight of cannabinoid(s), from 25 to 50% by weight phospholipids; from 18 to 50% by weight of therapeutically effective oil.

16. A composition according to claim 7, comprising at least one glycol and at least one vegetable oil.

17. A composition according to claim 16, comprising propylene glycol and pomegranate oil.

18. A composition for nasal administration according to claim 16, comprising from 25 to 40% by weight of cannabinoid(s), from 30 to 50% by weight phospholipids, from 10 to 30% of propylene glycol and from 3 to 20% by weight of vegetable oil.

19. A composition according to claim 7, comprising from 25 to 40% by weight of CBD, from 1 to 25% by weight of THC, from 30 to 45% of phospholipids, from 1 to 30% of propylene glycol and from 0.1 to 1.5 of antioxidant.

20. A composition according to claim 7, comprising from 25 to 35% by weight of CBD, from 1 to 15% by weight of THC, from 1 to 15% by weight of CBN, from 30 to 45% of phospholipids, from 1 to 25% of propylene glycol and from 0.1 to 1.5 of antioxidant.

21. A composition according to claim 7, comprising from 35 to 50% by weight one or more cannabinoids and from 35 to 50% by weight phospholipid(s).

22. A composition according to claim 21 for rectal or vaginal administration, wherein the composition is devoid of added liquid.

23. A method of treating a disease and/or condition in a patient, combatting the progress of a disease or relieving symptoms associated therewith, comprising intranasal, rectal or vaginal administration of a composition according to claim 1.

24. A method according to claim 23, comprising intranasal administration of a composition for administration into the nasal cavity of a patient, wherein the composition is an essentially non-aqueous composition comprising:
not less than 25% by weight of one or more cannabinoid(s);
from 25% to 55% by weight one or more phospholipid(s); and optionally one or more antioxidants.

25. A method according to claim 24, for treating brain and CNS diseases and conditions.

26. A method according to claim 24, wherein the disease and condition is selected from the group consisting of neurological disorder, muscular disturbances, ticks, insomnia, pain, anxiety, migraine, glioma, epilepsy, blastoglioma, cancer, acne, IBD, Chron's disease, loss of appetite, anxiety, distress, panic, tremor, multiple sclerosis, menopause, including symptoms associated with menopause, autism, dementia, Alzheimer, Parkinson, awakens, mood disorders, post-trauma, alcoholic and nonalcoholic fatty liver, hysteria, seizure and types of encephalopathy, including hepatic-encephalopathy, hepatic cancer and cirrhosis, menstrual pain and cramps, premenstrual pain, painful menstrual periods and vaginal mucosa inflammation.

27. A method according to claim 26, for relieving pain in said patient.

28. A method according to claim 23, comprising rectal administration of a composition for administration into the rectal cavity of a patient, wherein the composition is an essentially non-aqueous composition comprising:
not less than 25% by weight of one or more cannabinoid(s);
from 25% to 55% by weight one or more phospholipid(s); and optionally one or more antioxidants.

29. A method according to claim 28, for the treatment of liver disease selected from the group consisting of hepatic-encephalopathy, hepatic cancer and cirrhosis.
